(19) United States
(12) Patent Application Publication
(10) Pub. No.: US 2006/0191050 A1
(43) Pub. Date: Aug. 31, 2006

(54) Visor for Glasses

(76) Inventor: George Michael Papac, Pasadena, CA (US)

Correspondence Address: George M. Papac, 184 Ramona Place, Pasadena, CA, 91107, US

(21) Appl. No.: 10/906,595

(22) Filed: Feb. 25, 2005

(51) Int. Cl. A61F 9/00

(52) U.S. Cl. 2/12

(57) ABSTRACT

A sun visor for attachment to a pair of eyeglasses temples. The visor is a flat surface with an inner concave and a convex outer edge. The visor itself resembles the bill of a baseball cap. The visor is added to a pair of glasses by slipping the temples through the temple holes in the visor and sliding the visor to the front most part of the glasses. By dong so the visor will arch upward and due to the two temple holes the visor will remain in place giving shade from the sun to the wearer. There are two things unique about my visor design. The first thing that is unique about my sun visor is that it has two template holes which gives stability to the visor and does not allow the visor to fall forward into the wearers face. The second is the slits in the material connected to the template holes allowing templates that are thicker in the middle to slide forward to the front end of the template hinge.

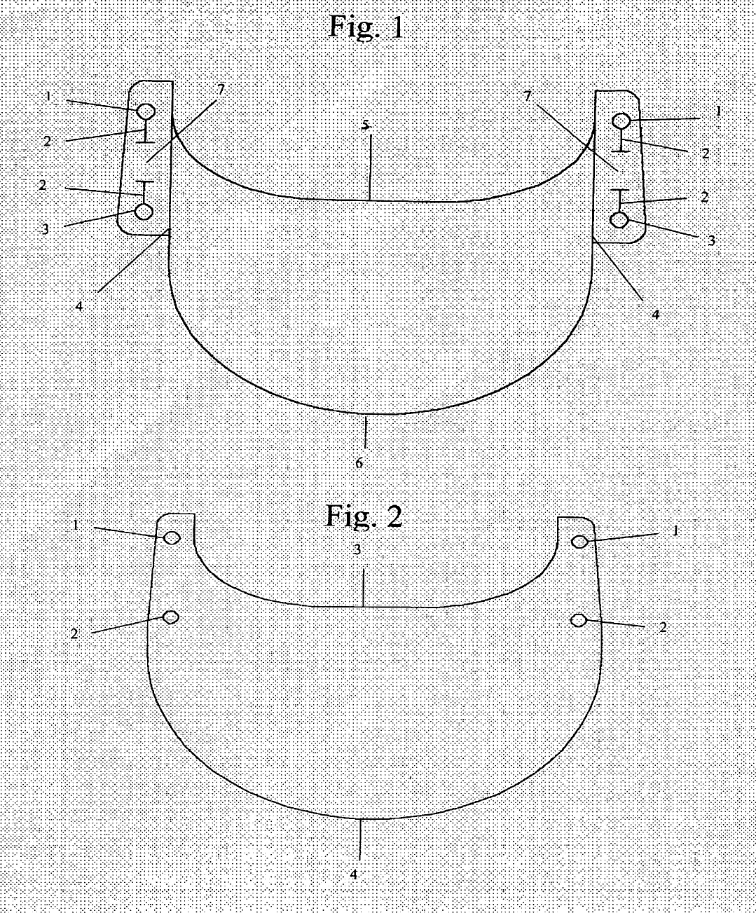

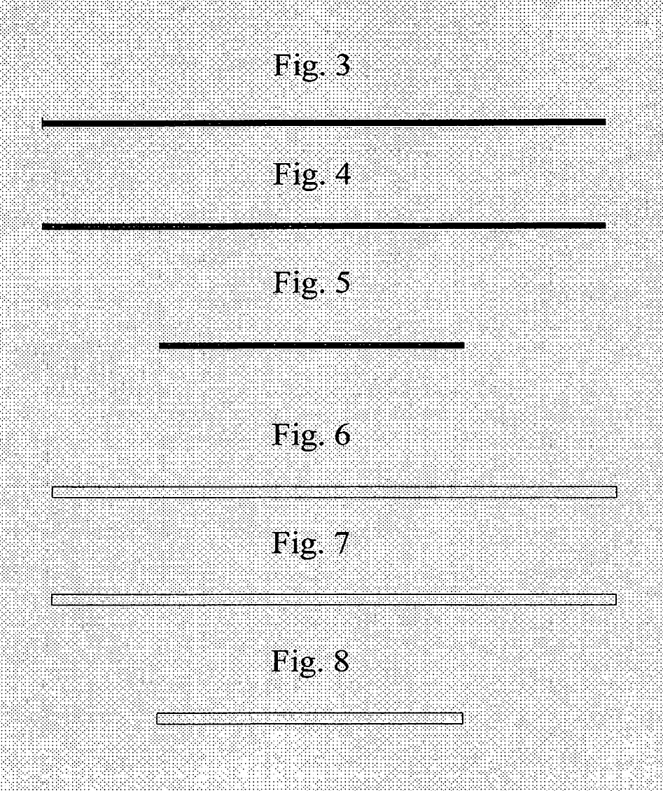

The VISOR can be made of but is not limited to paper, paper card stock, thin metal, Styrofoam, celluloid, foam sheeting, wood, plastic or any other material that is deemed suitable for its intended use. It can be made in any color, solid or opaque.

The VISOR is best described as a platform resembling the bill of a baseball cap. The VISOR bill, at the forward most end away from the face, can be rounded, pointed or any other shape or collection of shapes as long as it performs its intended use as a shade from the sun or rain.

The VISOR attaches to the templates of a pair of reading or sunglasses, and by doing so it provides shade from the sun to the wearer. The VISOR does not use any other peripherals to attach to the glasses.

1-5. (canceled)

6. The ornamental design for a sun visor for eyeglasses, as shown and described, what distinguishes my design from others is that it has two template holes so that the visor will not fall forward in the wearers face and obstruct the wearers vision, that the slits cut in the visor connecting to the template holes will accommodate larger temples while being slipped in to position for wearing.
